(19) United States
(12) Patent Application Publication
(10) Pub. No.: US 2012/0004292 A1
(43) Pub. Date: Jan. 5, 2012

(54) COMPOSITION FOR INJECTION COMPRISING AN INJECTION MEDICINAL PRODUCT AND A GEL

(76) Inventor: Alain Villette, Saint Pierre Des Echaubrognes (FR)

(21) Appl. No.: 13/174,809

(22) Filed: Jul. 1, 2011

(30) Foreign Application Priority Data
Jul. 5, 2010 (FR) 10 55 426

(51) Int. Cl. A61K 31/381; A61P 29/00

(52) U.S. Cl. 514/448

(57) ABSTRACT

A composition of an injectable medicinal product and a gel, concentrating the medicinal product on-site to reinforce its action and/or reduce the toxicity of the medicinal product.

### BACKGROUND

The majority of medicinal products for injection are in the form of liquids having equivalent fluidity to water. Some have oily viscosity. In addition, most medicinal products for injection have ambivalent activity, namely that they firstly have positive curative activity, which is the reason for use thereof, and secondly varying degrees of negative toxic activity which limits the use thereof. This ambivalence concerns all products for injection.

The present invention particularly concerns medicinal products having local action. These products have local as well as general toxicity with the result that some products which have uncontrolled toxicity are not used locally.

Any liquid product injected into the body is diluted in vascular, extracellular, and cellular biological liquids. This dilution is detrimental if local action of the injected product is desired, since it locally reduces the concentration of the active ingredient.

The user of the medicinal product is very often confronted with the following problems: increase the injected amount to obtain higher activity, but which will inevitably go hand-in-hand with an increase in toxicity limit which can be reached fairly rapidly.

The problem raised is, therefore, how to increase the power of a medicinal product without increasing the toxicity thereof?

This problem particularly arises in dental anaesthesia in which, to obtain the expected result, an active ingredient is added which is the anaesthetic molecule, a vasoactive agent, generally adrenaline.

Adrenaline is an endogenous catecholamine, hence permanently secreted by the human body.

This vasoactive agent, combined with an anaesthetizing liquid, leads to vasoconstriction of the vessels in the injected region.

Such vasoconstriction reduces the volume of the vessels, hence the blood volume; dilution is thereby reduced, which leads to an increase in the concentration of the active ingredient.

By slowing diffusion in the body, it reduces toxicity. Therefore, the presence of the vasoactive agent reinforces and extends the action of the anaesthetic and, at the same time, reduces the toxicity of the injection.

The local action of adrenaline may be harmful if its action is extended.

Adrenaline, when injected into poorly irrigated tissue, or in too great quantity, may lead to necrosis of the tissue.

From a general viewpoint, it is its hypotensive action, followed by hypertensive action, that is considered adverse which may lead to feelings of faintness (weak legs) and of tachycardia, ill-perceived by both patients and practitioners.

Practitioners also fear harmful effects on the heart.

Similarly, it is seen to carry problems (allergy in particular) related to the preserving and antioxidant agents which are added to adrenaline to ensure anti-degradation thereof.

The negative effects of adrenaline or noradrenaline, the chief vasoactive agents used, are described in the following documents:
Mitsuhiro Haraguchi US 2006/018 9572 A1,
Mitsuhiro Haraguchi US 2006/021 6245 A1,
Al Reader U.S. Pat. No. 6,075,059 A,
Mitsuhiro Haraguchi U.S. Pat. No. 6,008,256 A, and
Kim K. Forrest U.S. Pat. No. 4,963,345.

On reading these documents, it is seen that the first concern of the authors is partly to replace or entirely to remove catecholamines from dental anaesthesia solutions.

The reduced diffusion of the injected solution can be obtained by increasing the viscosity of the injected solution. This reduced diffusion is dependent upon the viscosity of the solution. The higher the viscosity of the solution, the more the diffusion thereof is limited. It is then advantageously possible to replace adrenaline by a gel.

### SUMMARY OF THE INVENTION

The present invention therefore proposes combining or assembling an anaesthetic with a gel, irrespective of the relative percentages of the two components and irrespective of the intended applications thereof.

### DETAILED DESCRIPTION

Adrenaline acts via chemical route, gel acts mechanically.

In addition to its mechanical action, a gel must meet certain criteria:
it must be biocompatible and non-pyrogenic,
it must not generate pain on injection, irrespective of the tissue density,
it must not prevent passing of the product through cortical bone, and
it must be fully absorbable.

Depending upon the viscosity of the solution, it will diffuse less far from the point of injection compared with an aqueous solution.

The feeling of numbness of soft tissue, unpleasantly perceived by patients, will be limited.

The gel incorporated in the anaesthetic solution, in the present invention, is an absorbable gel of sodium hyaluronidate, of animal or plant origin, which may or may not be cross-linked, or any other biocompatible gel.

In patent US 2006/018 9572 A1 to Mitsuhiro Haraguchi, the patentee claims the joint use of chondroitin sulphate and hydroxypropyl methylcellulose combined with lidocaine.

According to the present invention, only one type of gelling agent is used.

Hyaluronic acid, from which sodium hyaluronidate is produced, is a mucopolysaccharide acid like chondroitin sulphate.

This is the gel used in the clinical trials conducted.

The clinical trials entailed the preparation of solutions of lidocaine, articaine, mepivacaine, and prilocaine containing concentrations of anesthetizing molecules identical to those of solutions currently used, i.e., 2% lidocaine, 4% articaine, 3% mepivacaine, and 4% prilocaine.

To each solution, sodium hyaluronidate was added in various quantities to obtain different viscosities, allowing pain-free injections to be made.

The adjuvant used to complete the mixture to the proportion of 1.8 ml (the volume of an anaesthetic cartridge) was an isotonic solution of sodium chloride. It could have been just as possible to use a potassium salt.

Three viscosities were obtained, each adapted to an anaesthetic technique.

The viscosities obtained were measured with a strain-controlled rheometer and plate/plate geometry. The values given correspond to dynamic viscosity and are expressed in Pascals per second (PA/s).

The indicated values are not restrictive; they are only cited as examples to show that there are essentially three major families of products differing in viscosity whose order of magnitude is given below:

Viscosity I: 17.5 PA/s, pH 6.2, is obtained by assembling 1 ml of sodium hyaluronidate plus 0.8 ml of 9% articaine. This viscosity is intended for nerve trunks and soft tissues.

Viscosity II: 8.3 PA/s, pH 5.75, obtained by assembling 0.9 ml of sodium hyaluronidate plus 0.8 ml of articaine plus 0.1 milliliter of sodium chloride. This viscosity is intended for para-apical anaesthesia and optionally diploic bone anaesthesia in low density bone.

Viscosity III: 3.05 pH, 5.27, PA/S obtained by assembling 0.8 ml of sodium hyaluronidate plus 0.8 ml of articaine plus 0.2 ml of sodium chloride. This viscosity is intended for diploic and intraseptal anaesthesia.

These preparations can be prepared using lidocaine, mepivacaine and prilocaine in exactly the same proportions.

These preparations can be produced either in normal version of the gel or in cross-linked version. The cross-linked version prolongs the action of the anaesthetizing molecule.

The pH values obtained are globally 0.7 higher than those of conventional solutions, which improves the cytological toxicity of these novel solutions.

Another family of solutions was prepared by assembling the solutions presented above with added adrenaline to the proportion of 0.0050 mg per milliliter to obtain a 1:200 000 solution thereof, and 0.01 mg per milliliter to obtain a 1:100 000 solution thereof.

These solutions are intended to be used in extended surgery to reduce bleeding.

It could be said that minor vasoconstrictive action potentiates the mechanical action of the gel.

This addition of vasoactive agent can be made with all existing anaesthetic molecules.

Clinical Trials

These solutions were compared, in equal quantities, with conventional solutions without a vasoconstrictor (mepivacaine, lidocaine, articaine) and with 1:200 000 and 1:100 000 adrenaline for different anaesthesia techniques (para-apical, nerve trunk, intra-ligamentary, osteocentral, and transcortical).

For all the anaesthesia techniques, the duration of the anaesthesia obtained with the gelled solutions was twice the length of the duration obtained using solutions without vasoactive agent, and the equivalent 1:200 and 1:100 000 adrenaline solutions.

Comparative studies showed that it is possible to obtain exactly the same effect—same efficacy, same duration—when replacing adrenaline by a gel.

The results obtained in dental anaesthesia, namely: maintained anaesthetic effect equivalent to that of the most powerful current anaesthetic solutions, replacing the entirety of the catecholamines by absorbable gel, can be transposed to other medicinal products for injection.

The addition of a gel to an injection medicinal product having local action allows diffusion thereof to be limited, hence the on-site concentration thereof to be increased and general toxicity to be decreased.

This principle can be applied in numerous fields, in cases where the action of a product is to be targeted and where general toxicity via diffusion must be limited.

Example: treatment of a tumor by injection of a gelled product, with a view to sclerosing or destroying the tumor.

In this type of treatment, it is possible for the two effects to be combined, by adding a vasoconstrictor to the gelling product which will further limit diffusion of the product.

Cross-linking of the gel, of greater or lesser extent, leads to absorbability of greater or lesser duration.

The absorbability of the injected product, hence the action time thereof, can be modulated by cross-linking the gel to varying degrees.

1. A composition comprising an injection medicinal product and a gel, concentrating the medicinal product on-site and/or reducing toxicity of the medicinal product.

2. The composition according to claim 1, containing a gel of animal or plant origin.

3. The composition according to claim 1, having at least two degrees of cross-linking to improve absorbability.

4. The composition according to claim 1, having a viscosity chosen from 5.27 PA/s, 8.3 PA/s, and 17.5 PA/s.

5. A dental anesthesia including the composition of claim 1.

6. The composition according to claim 1 comprising at least one additional active ingredient and its adjuvants.

7. The composition according to claim 1 comprising a vasoactive agent.

8. The composition according to claim 1 comprising at least one preserving agent and/or at least one antioxidant.

9. The composition according to claim 2, having at least two degrees of cross-linking to improve absorbability.

10. A dental anesthesia including the composition of claim 2.

11. The composition according to claim 2 comprising a vasoactive agent.

12. The composition according to claim 2 comprising at least one preserving agent and/or at least one antioxidant.
